(19) United States
(12) Patent Application Publication
(10) Pub. No.: US 2023/0270504 A1
(43) Pub. Date: Aug. 31, 2023

(54) SYSTEM AND METHOD FOR PROSTHETIC PLACEMENT, SURGICAL GUIDE PLACEMENT, AND ORTHOGNATHIC SURGERY REASSEMBLY

(71) Applicant: NEOCIS INC., Miami, FL (US)

(72) Inventor: Alon Mozes, Miami Beach, FL (US)

(21) Appl. No.: 18/040,621

(22) PCT Filed: Aug. 4, 2021
(86) PCT No.: PCT/IB2021/057161

§ 371 Date: Feb. 3, 2023

Related U.S. Application Data
(60) Provisional application No. 63/062,162, filed on Aug. 6, 2020.

(51) Int. Cl. A61B 34/20; A61B 34/32; A61C 8/00

(52) U.S. Cl. CPC A61B 34/20; A61B 34/32; A61C 8/0089; A61B 2034/2051; A61B 2034/2055

(57) ABSTRACT

A robotic system and method involve a fiducial marker engaged with a maxillofacial anatomy, and an end effector at a distal end of an articulating arm member. The end effector receives a dental element comprising a prosthetic device, a surgical guide, or an orthognathic element, in a predetermined spatial relationship therewith. A controller device directly communicates with the fiducial marker, the articulating arm member, and the end effector, to determine an end effector disposition relative to the fiducial marker during end effector movement according to a virtual procedure plan for placing the dental element in aligned relation to the maxillofacial anatomy, and directs the articulating arm member to physically control allowable movement of the end effector, directly relative to the disposition of the end effector with respect to the fiducial marker and according to the virtual procedure plan, to place the dental element for securement to the maxillofacial anatomy.

### BACKGROUND

### Field of the Disclosure

The present application relates to dental robotic systems and, more particularly, to a system and method implementing a dental robot for prosthetic placement, surgical guide placement, and orthognathic surgery reassembly.

### Description of Related Art

Some dental robotic systems implement haptic guidance of a drill for drilling the osteotomy of a patient for receiving a dental implant. Such dental robotic systems can also implement haptic guidance for placing the implant anchor using the same drill with a different attachment (e.g., in place of a drill bit). However, the next step in restoring the patient's dentition is to place the prosthetic(s) into engagement with the implant anchor(s). Though the dental robotic system may be able to accurately aid in drilling the hole(s) in the osteotomy and then placing the implant anchor(s), there still exists a risk for error in placing the prosthetic(s) into engagement with the implant anchor(s).

More particularly, the prosthetic generally includes hole(s) to accommodate and receive the implant anchor(s) implanted in the patient so as to facilitate securement of the prosthetic to the implant anchor(s) and to complete the assembly. In some instances, the hole(s) in the prosthetic are often made large enough (e.g., oversized) to account for deviations in the location(s) of the implant anchor(s). However, the oversized holes in the prosthetic can result in the prosthetic to move or shift around or along the implant anchor(s). In this regard, it is often important to have relative alignment between an upper and lower arch in order to reproduce the proper bite occlusion. As such, it is important to ensure that the prosthetic/denture is secured to the implant anchor(s) precisely as planned. Similar needs also exist in instances where, for example, a surgical guide is to be attached to the patient, or in orthognathic surgery where an excised maxilla or portion of a maxilla must be reattached to the patient's skull.

Thus, there exists a need for a system and method for conducting prosthetic placement, surgical guide placement, and/or orthognathic surgery reassembly in which the prosthetic, surgical guide, or the maxilla can be more precisely placed and affixed to the patient's support structure (e.g., the implant anchor(s) or the skull structure) as compared to prior art procedures. Such a system and method should preferably be readily implemented and ergonomically friendly for the dental professional, and should desirably provide a convenient and effective training tool for the dental professional to develop their skills in regard to the noted procedures.

### SUMMARY OF THE DISCLOSURE

The above and other needs are met by aspects of the present disclosure which, in one particular aspect, provides a dental robotic system. Such a system includes a fiducial marker adapted to be engaged with maxillofacial anatomy and an articulating arm member having an end effector engaged with a distal end thereof. The end effector is arranged to receive a prosthetic device, a surgical guide, or an orthognathic element, in a predetermined spatial relationship with the end effector. A controller device has a processor and a memory storing a computer program product, wherein the computer program product is executable by the processor to perform the step of communicating with the fiducial marker, the articulating arm member, and the end effector, to determine a disposition of the end effector in relation to the fiducial marker during movement of the end effector in accordance with a virtual procedure plan for placing the prosthetic device, the surgical guide, or the orthognathic element in an aligned relation to the maxillofacial anatomy. The articulating arm member is also directed to physically control allowable movement of the end effector, directly relative to the disposition of the end effector with respect to the fiducial marker engaged with the maxillofacial anatomy, according to the virtual procedure plan, in order to place the prosthetic device, the surgical guide, or the orthognathic element in the aligned relation for securement to the maxillofacial anatomy.

Another aspect of the present disclosure provides a method of conducting a dental procedure implementing a dental robotic system. Such a method comprises engaging a fiducial marker with maxillofacial anatomy, and receiving a prosthetic device, a surgical guide, or an orthognathic element, in a predetermined spatial relationship with an end effector engaged with a distal end of an articulating arm member. A computer program product is executed with a processor of a controller device to perform the step of communicating with the fiducial marker, the articulating arm member, and the end effector, to determine a disposition of the end effector in relation to the fiducial marker during movement of the end effector in accordance with a virtual procedure plan for placing the prosthetic device, the surgical guide, or the orthognathic element in an aligned relation to the maxillofacial anatomy. The articulating arm member is also directed to physically control allowable movement of the end effector, directly relative to the disposition of the end effector with respect to the fiducial marker engaged with the maxillofacial anatomy, according to the virtual procedure plan, in order to place the prosthetic device, the surgical guide, or the orthognathic element in the aligned relation for securement to the maxillofacial anatomy.

The present disclosure thus includes, without limitation, the following example embodiments:

Example Embodiment 1: A dental robotic system, comprising a fiducial marker adapted to be engaged with maxillofacial anatomy; an articulating arm member having an end effector engaged with a distal end thereof, the end effector being arranged to receive a prosthetic device, a surgical guide, or an orthognathic element, in a predetermined spatial relationship with the end effector; and a controller device having a processor and a memory storing a computer program product executable by the processor to perform the steps of communicating with the fiducial marker, the articulating arm member, and the end effector, to determine a disposition of the end effector in relation to the fiducial marker during movement of the end effector in accordance with a virtual procedure plan for placing the prosthetic device, the surgical guide, or the orthognathic element in an aligned relation to the maxillofacial anatomy; and directing the articulating arm member to physically control allowable movement of the end effector, directly relative to the disposition of the end effector with respect to the fiducial marker engaged with the maxillofacial anatomy, according to the virtual procedure plan, in order to place the prosthetic device, the surgical guide, or the orthognathic element in the aligned relation for securement to the maxillofacial anatomy.

Example Embodiment 2: The system of any preceding example embodiment, or combinations thereof, wherein execution of the computer program product by the processor of the controller device causes the controller device to perform the step of directing the end effector to provide haptic feedback, if movement of the end effector deviates from the virtual procedure plan, as the prosthetic device, the surgical guide, or the orthognathic element is being placed in the aligned relation to the maxillofacial anatomy.

Example Embodiment 3: The system of any preceding example embodiment, or combinations thereof, comprising a splint device physically and securely interacted with the maxillofacial anatomy, and operably engaged with the fiducial marker.

Example Embodiment 4: The system of any preceding example embodiment, or combinations thereof, wherein the controller device or the end effector is arranged to provide audio feedback or visual feedback, if movement of the end effector deviates from the virtual procedure plan.

Example Embodiment 5: The system of any preceding example embodiment, or combinations thereof, wherein the prosthetic device, the surgical guide, or the orthognathic element is arranged to be directly received by the end effector in a predetermined spatial relationship therewith.

Example Embodiment 6: The system of any preceding example embodiment, or combinations thereof, comprising an interface received by the end effector in a predetermined spatial relationship therewith, the interface being arranged to receive the prosthetic device, the surgical guide, or the orthognathic element in a predetermined spatial relationship therewith.

Example Embodiment 7: The system of any preceding example embodiment, or combinations thereof, wherein the interface is a bite form appliance arranged to receive the prosthetic device in the predetermined spatial relationship therewith.

Example Embodiment 8: The system of any preceding example embodiment, or combinations thereof, wherein the step of directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the prosthetic device in the aligned relation with one or more implant anchors implanted in the maxillofacial anatomy to secure the prosthetic device thereto.

Example Embodiment 9: The system of any preceding example embodiment, or combinations thereof, wherein the step of directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the surgical guide in the aligned relation with the maxillofacial anatomy, the surgical guide defining one or more guide holes or having one or more guide inserts engaged therewith, with the one or more guide inserts each defining a guide hole, so as to allow securement holes to be drilled in the maxillofacial anatomy through the guides holes or to allow fasteners to be received through the guide holes for engagement with the securement holes so as to secure the surgical guide to the maxillofacial anatomy.

Example Embodiment 10: The system of any preceding example embodiment, or combinations thereof, wherein the prosthetic device defines one or more guide holes, or has one or more guide inserts engaged therewith, with the one or more guide inserts each defining a guide hole, such that the prosthetic device is the surgical guide.

Example Embodiment 11: The system of any preceding example embodiment, or combinations thereof, wherein the orthognathic element is a portion of the maxillofacial anatomy separated from a remainder of the maxillofacial anatomy, and wherein the step of directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the portion of the maxillofacial anatomy in the aligned relation with the remainder of the maxillofacial anatomy, so as to allow securement holes to be drilled in the portion or the remainder of the maxillofacial anatomy or to allow fasteners to be engaged with the securement holes so as to secure a bracket between the portion and the remainder of the maxillofacial anatomy.

Example Embodiment 12: The system of any preceding example embodiment, or combinations thereof, comprising a detector engaged with a distal end of a tracking arm, the tracking arm and the detector being in communication with the controller device, the detector being arranged in a spaced-apart relationship with the fiducial marker to detect the fiducial marker and cooperate with the controller device to determine a spatial relationship with the fiducial marker.

Example Embodiment 13: The system of any preceding example embodiment, or combinations thereof, wherein the detector is an electrical detector, an electromechanical detector, an electromagnetic detector, an optical detector, an infrared detector, or combinations thereof.

Example Embodiment 14: The system of any preceding example embodiment, or combinations thereof, comprising a tracking arm having a distal end physically engaged with the fiducial marker, the tracking arm being in communication with the controller device and arranged to cooperate with the controller device to determine a spatial relationship with the fiducial marker.

Example Embodiment 15: The system of any preceding example embodiment, or combinations thereof, wherein the fiducial marker is arranged to be in communication with the controller device via an electrical communication system, a mechanical communication system, an electromechanical communication system, an electromagnetic communication system, an optical communication system, an infrared communication system, or combinations thereof.

Example Embodiment 16: The system of any preceding example embodiment, or combinations thereof, wherein the fiducial marker is arranged to be in communication with the controller device via a wireless communication system or a wired communication system.

Example Embodiment 17: The system of any preceding example embodiment, or combinations thereof, wherein the controller device is arranged to facilitate graphical manipulation of an image of the maxillofacial anatomy to form the virtual procedure plan for the maxillofacial anatomy receiving the prosthetic device, the surgical guide, or the orthognathic element in the aligned relation therewith.

Example Embodiment 18: A method of conducting a dental procedure implementing a dental robotic system, the method comprising engaging a fiducial marker with maxillofacial anatomy; receiving a prosthetic device, a surgical guide, or an orthognathic element, in a predetermined spatial relationship with an end effector engaged with a distal end of an articulating arm member; and executing a computer program product with a processor of a controller device to perform the steps of communicating with the fiducial marker, the articulating arm member, and the end effector, to determine a disposition of the end effector in relation to the fiducial marker during movement of the end effector in accordance with a virtual procedure plan for placing the prosthetic device, the surgical guide, or the orthognathic element in an aligned relation to the maxillofacial anatomy; and directing the articulating arm member to physically control allowable movement of the end effector, directly relative to the disposition of the end effector with respect to the fiducial marker engaged with the maxillofacial anatomy, according to the virtual procedure plan, in order to place the prosthetic device, the surgical guide, or the orthognathic element in the aligned relation for securement to the maxillofacial anatomy.

Example Embodiment 19: The method of any preceding example embodiment, or combinations thereof, wherein executing the computer program product with the processor of the controller device causes the controller device to perform the step of directing the end effector to provide haptic feedback, if movement of the end effector deviates from the virtual procedure plan, as the prosthetic device, the surgical guide, or the orthognathic element is being placed in the aligned relation to the maxillofacial anatomy.

Example Embodiment 20: The method of any preceding example embodiment, or combinations thereof, wherein engaging a fiducial marker with the maxillofacial anatomy comprises physically and securely interacting a splint device with the maxillofacial anatomy, the splint device being operably engaged with the fiducial marker.

Example Embodiment 21: The method of any preceding example embodiment, or combinations thereof, comprising arranging the controller device or the end effector to provide audio feedback or visual feedback, if movement of the end effector deviates from the virtual procedure plan.

Example Embodiment 22: The method of any preceding example embodiment, or combinations thereof, comprising directly receiving the prosthetic device, the surgical guide, or the orthognathic element with the end effector, such that the prosthetic device, the surgical guide, or the orthognathic element is received in a predetermined spatial relationship therewith.

Example Embodiment 23: The method of any preceding example embodiment, or combinations thereof, comprising receiving an interface with the end effector in a predetermined spatial relationship therewith, the interface being arranged to receive the prosthetic device, the surgical guide, or the orthognathic element in a predetermined spatial relationship therewith.

Example Embodiment 24: The method of any preceding example embodiment, or combinations thereof, wherein the interface is a bite form appliance and wherein the method comprises receiving the prosthetic device with the bite form appliance in the predetermined spatial relationship therewith.

Example Embodiment 25: The method of any preceding example embodiment, or combinations thereof, wherein directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the prosthetic device in the aligned relation with one or more implant anchors implanted in the maxillofacial anatomy to secure the prosthetic device thereto.

Example Embodiment 26: The method of any preceding example embodiment, or combinations thereof, wherein directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the surgical guide in the aligned relation with the maxillofacial anatomy, the surgical guide defining one or more guide holes or having one or more guide inserts engaged therewith, with the one or more guide inserts each defining a guide hole, so as to allow securement holes to be drilled in the maxillofacial anatomy through the guides holes or to allow fasteners to be received through the guide holes for engagement with the securement holes so as to secure the surgical guide to the maxillofacial anatomy.

Example Embodiment 27: The method of any preceding example embodiment, or combinations thereof, comprising forming one or more guide holes in the prosthetic device, or engaging one or more guide inserts with the prosthetic device, with the one or more guide inserts each defining a guide hole, such that the prosthetic device becomes the surgical guide.

Example Embodiment 28: The method of any preceding example embodiment, or combinations thereof, wherein the orthognathic element is a portion of the maxillofacial anatomy separated from a remainder of the maxillofacial anatomy, and wherein directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the portion of the maxillofacial anatomy in the aligned relation with the remainder of the maxillofacial anatomy, so as to allow securement holes to be drilled in the portion or the remainder of the maxillofacial anatomy or to allow fasteners to be engaged with the securement holes so as to secure a bracket between the portion and the remainder of the maxillofacial anatomy.

Example Embodiment 29: The method of any preceding example embodiment, or combinations thereof, wherein communicating with the fiducial marker comprises communicating with the fiducial marker via a detector engaged with a distal end of a tracking arm, the tracking arm and the detector being in communication with the controller device, and the detector being arranged in a spaced-apart relationship with the fiducial marker to detect the fiducial marker and cooperate with the controller device to determine a spatial relationship with the fiducial marker.

Example Embodiment 30: The method of any preceding example embodiment, or combinations thereof, wherein communicating with the fiducial marker comprises communicating with the fiducial marker via an electrical detector, an electromechanical detector, an electromagnetic detector, an optical detector, an infrared detector, or combinations thereof, engaged with the distal end of the tracking arm.

Example Embodiment 31: The method of any preceding example embodiment, or combinations thereof, wherein communicating with the fiducial marker comprises communicating with the fiducial marker via a distal end of a tracking arm, the distal end being physically engaged with the fiducial marker, the tracking arm being in communication with the controller device and arranged to cooperate with the controller device to determine a spatial relationship with the fiducial marker.

Example Embodiment 32: The method of any preceding example embodiment, or combinations thereof, comprising communicating between the fiducial marker and the controller device via an electrical communication system, a mechanical communication system, an electromechanical communication system, an electromagnetic communication system, an optical communication system, an infrared communication system, or combinations thereof.

Example Embodiment 33: The method of any preceding example embodiment, or combinations thereof, comprising communicating between the fiducial marker and the controller device via a wireless communication system or a wired communication system.

Example Embodiment 34: The method of any preceding example embodiment, or combinations thereof, comprising graphically manipulating an image of the maxillofacial anatomy with the controller device to form the virtual procedure plan for the maxillofacial anatomy receiving the prosthetic device, the surgical guide, or the orthognathic element in the aligned relation therewith.

These and other features, aspects, and advantages of the present disclosure will be apparent from a reading of the following detailed description together with the accompanying drawings, which are briefly described below. The present disclosure includes any combination of two, three, four, or more features or elements set forth in this disclosure, regardless of whether such features or elements are expressly combined or otherwise recited in a specific embodiment description herein. This disclosure is intended to be read holistically such that any separable features or elements of the disclosure, in any of its aspects and embodiments, should be viewed as intended, namely to be combinable, unless the context of the disclosure clearly dictates otherwise.

It will be appreciated that the summary herein is provided merely for purposes of summarizing some example aspects so as to provide a basic understanding of the disclosure. As such, it will be appreciated that the above described example aspects are merely examples and should not be construed to narrow the scope or spirit of the disclosure in any way. It will be appreciated that the scope of the disclosure encompasses many potential aspects, some of which will be further described below, in addition to those herein summarized. Further, other aspects and advantages of such aspects disclosed herein will become apparent from the following detailed description taken in conjunction with the accompanying drawings which illustrate, by way of example, the principles of the described aspects.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIGS. 1-3 schematically illustrate a dental robotic system, according to one aspect of the present disclosure;

FIG. 4 schematically illustrates a dental robotic system, according to another aspect of the present disclosure;

FIG. 5 schematically illustrates a dental robotic system according to one aspect of the present disclosure being applied in the placement of a prosthetic device;

FIGS. 6A and 6B schematically illustrate a dental robotic system according to another aspect of the present disclosure being applied in the placement of a prosthetic device;

FIGS. 7A and 7B schematically illustrate a dental robotic system according to one aspect of the present disclosure being applied in the placement of a surgical guide; and

FIGS. 8A and 8B schematically illustrates a dental robotic system according to one aspect of the present disclosure being applied in the placement of an orthognathic element.

### DETAILED DESCRIPTION OF THE DISCLOSURE

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein; rather, these aspects are provided so that this disclosure will satisfy applicable legal requirements. Like numbers refer to like elements throughout.

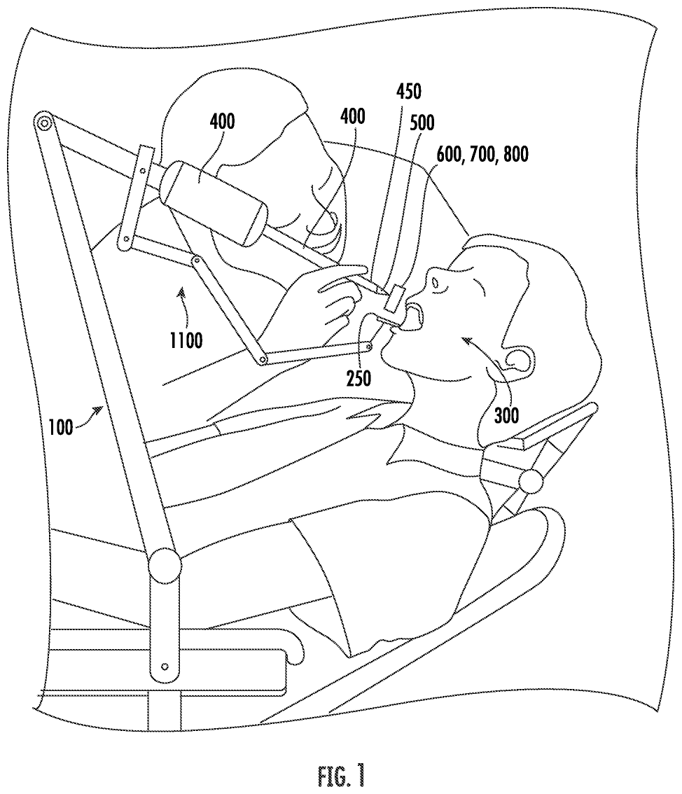

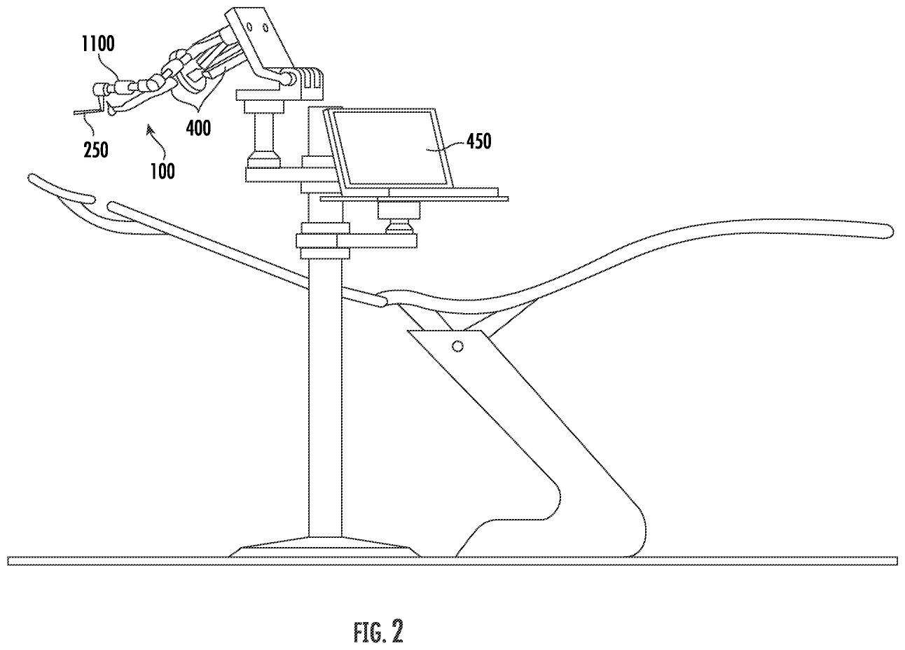

FIGS. 1-4 illustrate various aspects of a dental robotic system, generally indicated by the numeral 100. Such a system 100 includes a fiducial marker 250 adapted to be engaged with maxillofacial anatomy 300. In some aspects, the system 100 further includes an articulating arm member 400 having an end effector 500 engaged with a distal end 450 of the articulating arm member 400. The end effector 500 is arranged to receive a prosthetic device 600, a surgical guide 700, or an orthognathic element 800, in a predetermined spatial relationship with the end effector 500 (e.g., the spatial relation between the end effector 500 and any of the prosthetic device 600, surgical guide 700, or orthognathic element 800 received by the end effector 500 is known). A controller device 450 has a processor and a memory storing a computer program product executable by the processor. The controller device 450 is arranged in communication with the fiducial marker 250.

While aspects of the present disclosure are described in terms of the maxillofacial anatomy and orthognathic element(s) thereof, a person of ordinary skill in the art will appreciate that the maxillofacial anatomy and the orthognathic element(s), in some aspects, are directed and refer to non-human models or other non-human representations or reproductions of such anatomy wherein the disclosed systems and methods herein are implemented to provide a convenient and effective training tool or training provision for the dental professional to develop their skills in regard to the procedures described herein. Moreover, the methods disclosed and claimed herein are particularly directed to the control and operation of the systems described and claimed herein, wherein such methods are not particularly directed to methods of surgery on humans.

In some aspects, upon execution of the computer program product/software by the processor, the controller device 450 is configured/arranged to communicate with the fiducial marker 250, the articulating arm member 400, and the end effector 500, so as to determine a disposition (or spatial relationship) of the end effector 500 in relation to the fiducial marker 250, particularly during movement of the end effector 500 in accordance with a virtual procedure plan for placing the prosthetic device 600, the surgical guide 700, or the orthognathic element 800 in an aligned relation to/with the maxillofacial anatomy 300. In such aspects, the controller device 450 is arranged to facilitate graphical manipulation of an image of the maxillofacial anatomy 300 to form the virtual procedure plan for the maxillofacial anatomy 300, whether the maxillofacial anatomy 300 is arranged to receive the prosthetic device 600, the surgical guide 700, or the orthognathic element 800 in the aligned relation therewith.

Upon determination of the disposition/spatial relation of the end effector 500 in relation to the fiducial marker 250, execution of the computer program product/software by the processor causes the controller device 450 to direct the articulating arm member 400 to physically control allowable movement of the end effector 500, directly relative to the disposition (or spatial relation) of the end effector 500 with respect to the fiducial marker 250 engaged with the maxillofacial anatomy 300, according to the virtual procedure plan, in order to place the prosthetic device 600, the surgical guide 700, or the orthognathic element 800 in the aligned relation for securement to the maxillofacial anatomy 300.

In some aspects, execution of the computer program product by the processor of the controller device 450 causes the controller device 450 to perform the step of directing the end effector 500 to provide haptic feedback, if movement of the end effector 500 deviates from the virtual procedure plan, as the prosthetic device 600, the surgical guide 700, or the orthognathic element 800 is being placed in the aligned relation to/with the maxillofacial anatomy 300. In the alternative, or in addition to, the end effector 500 providing haptic feedback, the controller device 450 or the end effector 500 can also be arranged to provide audio feedback (e.g., via a sound-emitting device) or visual feedback (e.g., via a display), if movement of the end effector 500 deviates from the virtual procedure plan.

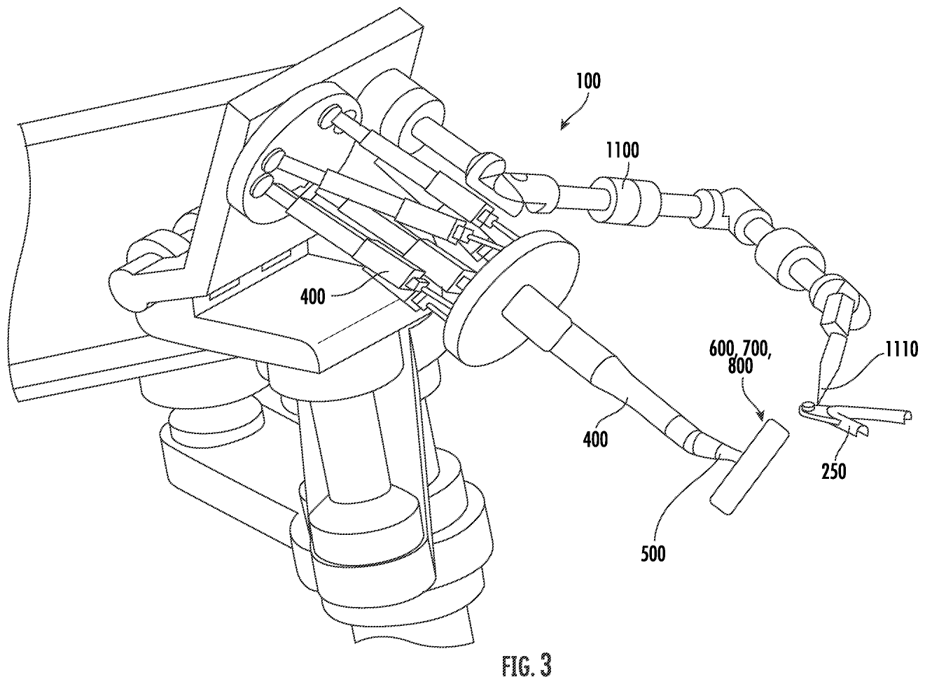

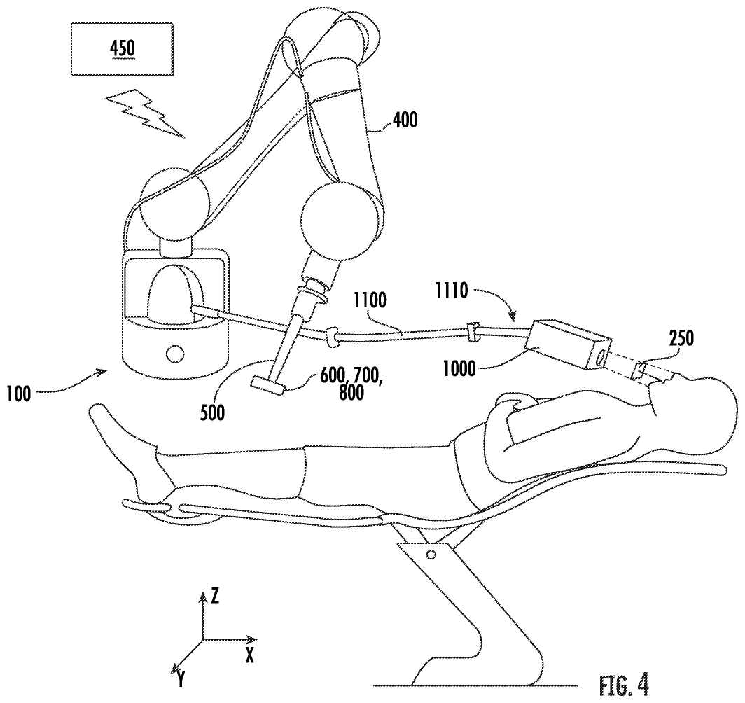

The fiducial marker 250, in some aspects, is adapted to be engaged with the maxillofacial anatomy 300 (e.g., as a splint device physically and securely interacted with the maxillofacial anatomy 300). In other aspects, the engagement between the splint device and the maxillofacial anatomy 300 forms the fiducial marker 250, or the fiducial marker 250 is operably engaged with the splint device in a known spatial relationship. The controller device 450 is further arranged to communicate with the fiducial marker 250 in different manners. For example, in one aspect as shown, for example, in FIG. 4, a detector 1000 is engaged with a distal end 1110 of a tracking arm 1100, and the tracking arm 1100 and the detector 1000 are in communication with the controller device 450. In such instances, the detector 1000 is arranged in a spaced-apart relationship (e.g., physically separated with a defined space therebetween) with the fiducial marker 250. The detector 1000 is arranged to detect the fiducial marker 250 and to cooperate with the controller device 450 to determine a spatial relationship with the fiducial marker 250 (e.g., the physical coordinates of the tracking arm 1100 and the detector 1000 with respect to the fiducial marker 250 are known by the controller 900). The detector 1000, in various aspects, is an electrical detector, an electromechanical detector, an electromagnetic detector, an optical detector, an infrared detector, or any other appropriate detector or combinations thereof.

In other aspects (see, e.g., FIGS. 1-3), the distal end 1110 of the tracking arm 1100 is physically engaged with the fiducial marker 250. In turn, the tracking arm 1100 is in communication with the controller device 450 and is arranged to cooperate with the controller device 450 to determine a spatial relationship with the fiducial marker 250 (e.g., the physical coordinates of the tracking arm 1100 and the fiducial marker 250 are known by the controller device 450). In these and other aspects, the fiducial marker 250 is arranged to be in communication with the controller device 450, for example, via an electrical communication system, a mechanical communication system, an electromechanical communication system, an electromagnetic communication system, an optical communication system, an infrared communication system, or combinations thereof. As such, the fiducial marker 250 is arranged to be in communication with the controller device 450 via a wireless communication system or a wired communication system.

In particular aspects, the prosthetic device 600, the surgical guide 700, or the orthognathic element 800 is arranged to be directly received by the end effector 500 in a predetermined spatial relationship therewith. That is, since the spatial relationship between the end effector 500 and the articulating arm member 400 is known by the controller device 450, the spatial relationship between the end effector 500 and the prosthetic device 600, the surgical guide 700, or the orthognathic element 800 directly received thereby is also known or readily determined by the controller device 450. As such, since the spatial relationship of the articulating arm member 400, the end effector 500, and the prosthetic device 600, the surgical guide 700, or the orthognathic element 800 is known to the controller device 450, and likewise the spatial relationship of the tracking arm 1100 and fiducial marker 250 also known to the controller device 450, a common coordinate system can be determined and established by the controller device 450 based on the fiducial marker 250.

Since the maxillofacial anatomy 300 can and, in some instances, does move during the procedure, the resulting movement of the fiducial marker 250 can be determined by the controller device 450, and thus the spatial relationship of the end effector 500 and the prosthetic device 600, the surgical guide 700, or the orthognathic element 800 can be adjusted for the movement of the fiducial marker 250 as the basis of the common coordinate system. As such, since the fiducial marker 250 is affixed to the maxillofacial anatomy 300, to which the prosthetic device 600, the surgical guide 700, or the orthognathic element 800 are intended to be engaged, precise and accurate alignment is provided between the maxillofacial anatomy 300 and the prosthetic device 600, the surgical guide 700, or the orthognathic element 800.

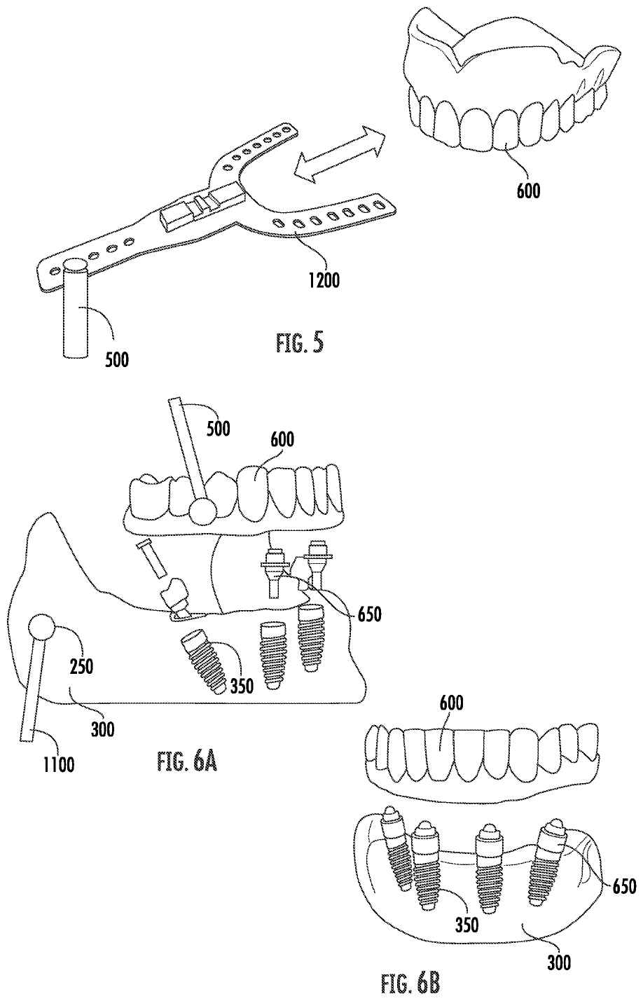

In some aspects, the end effector 500 is arranged to receive an interface 1200 (see, e.g., FIG. 5) in a predetermined spatial relationship therewith, wherein the interface 1200 is arranged to receive the prosthetic device 600, the surgical guide 700, or the orthognathic element 800 in a predetermined spatial relationship therewith. That is, since the spatial relationship between the end effector 500 and the articulating arm member 400 is known by the controller device 450, the spatial relationship between the end effector 500, the interface 1200 received thereby, and the prosthetic device 600, the surgical guide 700, or the orthognathic element 800 received by the interface 1200 is also known or readily determined by the controller device 450. In one example aspect, the interface 1200 is a bite fork appliance (see, e.g., FIG. 5) arranged to receive the prosthetic device 600 in the predetermined spatial relationship therewith.

In one aspect of the disclosure, the controller device 450 is arranged to direct the articulating arm member 400 to physically control allowable movement of the end effector 500, directly relative to the disposition of the end effector 500 with respect to the fiducial marker 250 engaged with the maxillofacial anatomy 300, and according to the virtual procedure plan, in order to place the prosthetic device 600 in the aligned relation with one or more implant anchors 650 implanted in the maxillofacial anatomy 300 to secure the prosthetic device 600 thereto (see, e.g., FIGS. 6A and 6B). The end effector 500 can have a drill mounted thereto, wherein the drill is modified to attach to the prosthetic device 600 (e.g., denture) using an appliance like a bite fork extending between the drill and the prosthetic device 600. In this manner, the drill and/or the bite fork can be considered an interface 1200. In another example, the prosthetic device 600 is mounted directly to the end effector 500, without the drill and/or the bite fork therebetween.

The implant anchors 650, whether manually implanted or implanted in a precise and accurate manner with the dental implantation system 100 as disclosed herein, are or can be readily determined by the controller device 450 relative to the fiducial marker 250 (e.g., through image analysis in preparation of the virtual procedure plan or from the actual implantation process for the implant anchors 650). Accordingly, receptacles for receiving the implant anchors 650, as defined by the prosthetic device 600, can be precisely and accurately formed both in size and in pattern to interface with the implant anchors 650, instead of the receptacles being oversized to allow for adjustments in place.

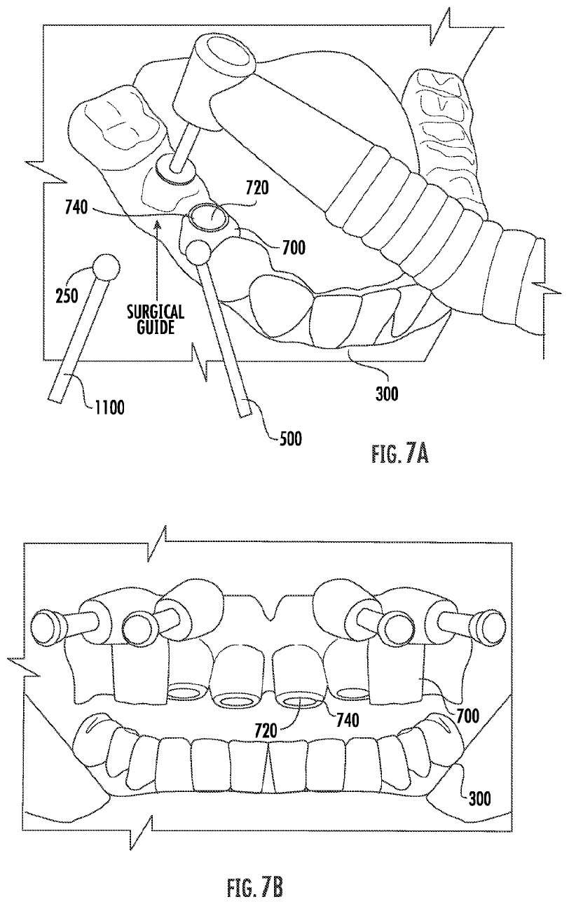

In another aspect of the disclosure, the controller device 450 is arranged to direct the articulating arm member 400 to physically control allowable movement of the end effector 500, directly relative to the disposition of the end effector 500 with respect to the fiducial marker 250 engaged with the maxillofacial anatomy 300, and according to the virtual procedure plan, in order to place the surgical guide 700 in the aligned relation with the maxillofacial anatomy 300. The surgical guide 700 (see, e.g., FIGS. 7A and 7B) defines one or more guide holes 720 or has one or more guide inserts 740 engaged therewith, wherein the one or more guide inserts 740 each define a guide hole 720. The surgical guide 700 thus allows securement holes 350 to be drilled in the maxillofacial anatomy 300 through the guides holes 720, or allows fasteners 760 to be received through the guide holes 720 for engagement with the securement holes 350 so as to secure the surgical guide 700 to the maxillofacial anatomy 300.

In some aspects, as will be understood by one skilled in the art, the prosthetic device 600 defines one or more guide holes 720, or has one or more guide inserts 740 engaged therewith, with the one or more guide inserts 740 each defining a guide hole 720, such that the prosthetic device 600 becomes a surgical guide 700, or otherwise is the surgical guide 700. That is, in some instances, the prosthetic device 600 can also function as a surgical guide that can be held in an appropriate position with respect to the maxillofacial anatomy 300 by the articulating arm member 400/end effector 500. Since the prosthetic device 600 will also be secured in relation to the securement hole 350, it is important to place the guiding prosthetic device 600 accurately relative to the maxillofacial anatomy 300. Such an arrangement can, for example, replace reliance on existing bone and/or anchor pins (see, e.g., FIG. 7B) for placing prior art surgical guides.

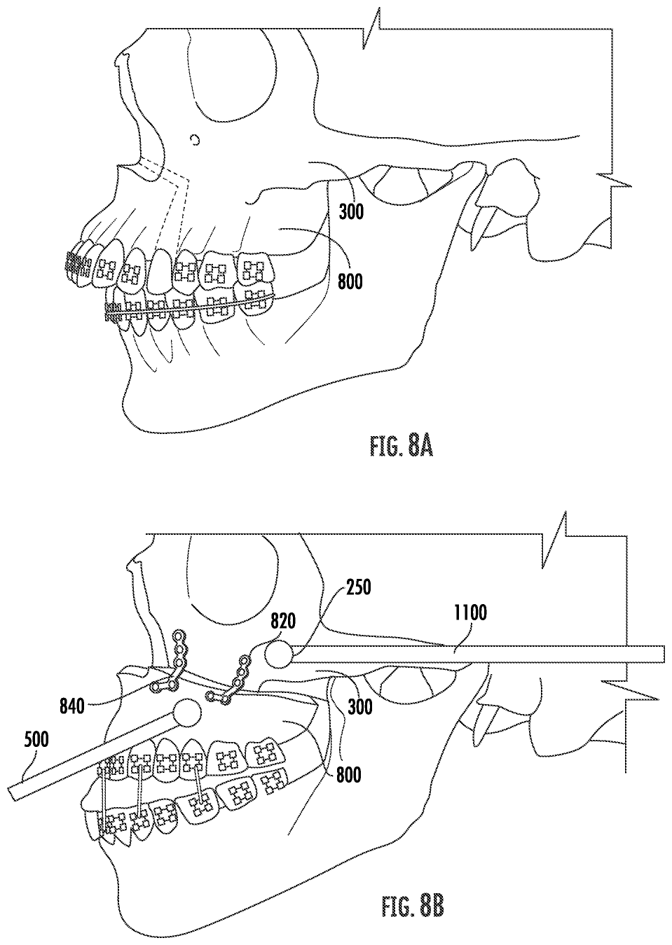

The orthognathic element 800 is a portion of the maxillofacial anatomy separated from a remainder of the maxillofacial anatomy 300 (see, e.g., FIGS. 8A and 8B). In still other aspects, the controller device 450 is arranged to direct the articulating arm member 400 to physically control allowable movement of the end effector 500, directly relative to the disposition of the end effector 500 with respect to the fiducial marker 250 engaged with the maxillofacial anatomy 300, and according to the virtual procedure plan, in order to place the portion of the maxillofacial anatomy (the orthognathic element 800) in the aligned relation with the remainder of the maxillofacial anatomy 300. Having the orthognathic element 800 aligned with the remainder of the maxillofacial anatomy 300 in this manner thus allows securement holes to be drilled in the portion of the maxillofacial anatomy (orthognathic element 800) or the remainder of the maxillofacial anatomy 300 and/or to allow fasteners 820 to be engaged with the securement holes so as to secure a bracket 840 between the portion of the maxillofacial anatomy (orthognathic element 800) and the remainder of the maxillofacial anatomy 300 such that the orthognatic element 800 is secured in the planned alignment with the remainder of the maxillofacial anatomy 300 (see, e.g., FIG. 8B).

In one example type of orthognathic surgery focused on the maxilla, a portion of the maxilla including the teeth is separated from the rest of the skull. It is then reattached in a desired alignment using plates and screws. The reattachment must be accurate in order to reproduce the intended anatomical shape and occlusion with the opposing arch. Implementing the articulating arm member 400/end effector 500 in the disclosed manner to securely hold the anatomical portion being separated from the skull, the tracking portion of the system 100 allows maxillofacial anatomy location to be monitored (e.g., via a fiducial marker 250) by communication with a portion of the skull not separated. The controller device 450 thus guides the articulating arm member 400/end effector 500 in placing the separated portion in relation to the skull for reattachment. The plates and screws for securing the separated portion can be drilled in situ at the proper/desired location while the articulating arm member 400/end effector 500 holds the separated anatomy in the right position relative to the skull.

With the spatial relationship in regard to the maxillofacial anatomy 300 established and known by the system 100, and the virtual procedure plan developed through the controller device 450, the alignment procedure for the prosthetic device 600, the surgical guide 700, or the orthognathic element 800 can then be initiated by the practitioner moving the end effector 500 toward the maxillofacial anatomy 300. In such instances, the controller device 450 is configured to control the movement of the end effector 500 via the articulating arm member 400 such that the action of the practitioner merely moves the end effector 500 to the appropriate starting position for the alignment procedure, with respect to the maxillofacial anatomy 300, as determined by the controller device 450 and dictated by the virtual procedure plan. Once the end effector 500 having the prosthetic device 600, the surgical guide 700, or the orthognathic element 800 engaged therewith is in the position dictated by the controller device 450, the active alignment portion of the actual procedure can then be initiated, wherein the controller device 450 may further dictate other parameters of the end effector 500 such as, for example, the position and orientation of the prosthetic device 600, the surgical guide 700, or the orthognathic element 800, also according to the virtual procedure plan.

In these instances, one distinction of the system 100 disclosed herein is that the end effector 500 having the prosthetic device 600, the surgical guide 700, or the orthognathic element 800 engaged therewith is not guided by the practitioner, but is only urged by the practitioner along a procedural route determined via the virtual procedure plan and implemented via the controller device 450 and the articulating arm member 400. That is, the system 100 may be configured to restrict the practitioner to performing the alignment procedure with respect to the maxillofacial anatomy, as determined via the virtual procedure plan and implemented via the controller device 450 and the articulating arm member 400, whereby the controller device 450 controls the allowable movement of the articulating arm member 400 (and thus the end effector 500) in accordance with the virtual procedure plan created from the image(s) of the maxillofacial anatomy. For instance, the system 100 may be configured for restricted movement of the articulating arm member 400/end effector 500, as communicated to the practitioner through tactile feedback, where, for example, the articulating arm member 400/end effector 500 may be easier to move according to the virtual procedure plan, and more difficult to move if deviating from the virtual procedure plan.

One skilled in the art will also appreciate, however, that the physical structure of the articulating arm member 400/end effector 500 to provide fully controlled movement according to the virtual procedure plan (i.e., due to vibration, flexing of components, and/or excessive force applied by the practitioner) and, as such, the system 100 may be further configured to provide other manners of feedback to the practitioner such as, for example, via a deviation warning indicia or any other suitable audio and/or visual mechanism. Therefore, the system 100 includes provisions for actually implementing the virtual procedure plan, and thus facilitates a more accurate alignment procedure, rather than merely warning the practitioner if any procedural parameters may be inaccurate. One skilled in the art will also appreciate, however, that, in some instances, the system 100 may be further configured to autonomously accomplish the virtual procedure plan, without the manipulation of the practitioner, through automatic manipulation of the articulating arm member 400/end effector 500 via the controller device 450.

Many modifications and other embodiments of the inventions set forth herein will come to mind to one skilled in the art to which these disclosed embodiments pertain having the benefit of the teachings presented in the foregoing descriptions and the associated drawings. Therefore, it is to be understood that embodiments of the invention are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the disclosure. In this regard, for example, different combinations of elements and/or functions than those explicitly described above are also contemplated within the scope of the disclosure. Although specific terms are employed herein, they are used in a generic and descriptive sense only and not for purposes of limitation.

It should be understood that although the terms first, second, etc. may be used herein to describe various steps or calculations, these steps or calculations should not be limited by these terms. These terms are only used to distinguish one operation or calculation from another. For example, a first calculation may be termed a second calculation, and, similarly, a second step may be termed a first step, without departing from the scope of this disclosure. As used herein, the term “and/or” and the “I” symbol includes any and all combinations of one or more of the associated listed items.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

That which is claimed:

1. A dental robotic system, comprising:
a fiducial marker adapted to be engaged with maxillofacial anatomy;
an articulating arm member having an end effector engaged with a distal end thereof, the end effector being arranged to receive a prosthetic device, a surgical guide, or an orthognathic element, in a predetermined spatial relationship with the end effector; and
a controller device having a processor and a memory storing a computer program product executable by the processor to perform the steps of:
communicating with the fiducial marker, the articulating arm member, and the end effector, to determine a disposition of the end effector in relation to the fiducial marker during movement of the end effector in accordance with a virtual procedure plan for placing the prosthetic device, the surgical guide, or the orthognathic element in an aligned relation to the maxillofacial anatomy; and
directing the articulating arm member to physically control allowable movement of the end effector, directly relative to the disposition of the end effector with respect to the fiducial marker engaged with the maxillofacial anatomy, according to the virtual procedure plan, in order to place the prosthetic device, the surgical guide, or the orthognathic element in the aligned relation for securement to the maxillofacial anatomy.

2. The system of claim 1, wherein execution of the computer program product by the processor of the controller device causes the controller device to perform the step of directing the end effector to provide haptic feedback, if movement of the end effector deviates from the virtual procedure plan, as the prosthetic device, the surgical guide, or the orthognathic element is being placed in the aligned relation to the maxillofacial anatomy.

3. The system of claim 1, comprising a splint device physically and securely interacted with the maxillofacial anatomy, and operably engaged with the fiducial marker.

4. The system of claim 1, wherein the controller device or the end effector is arranged to provide audio feedback or visual feedback, if movement of the end effector deviates from the virtual procedure plan.

5. The system of claim 1, wherein the prosthetic device, the surgical guide, or the orthognathic element is arranged to be directly received by the end effector in a predetermined spatial relationship therewith.

6. The system of claim 1, comprising an interface received by the end effector in a predetermined spatial relationship therewith, the interface being arranged to receive the prosthetic device, the surgical guide, or the orthognathic element in a predetermined spatial relationship therewith.

7. The system of claim 6, wherein the interface is a bite form appliance arranged to receive the prosthetic device in the predetermined spatial relationship therewith.

8. The system of claim 1, wherein the controller device step of directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the prosthetic device in the aligned relation with one or more implant anchors implanted in the maxillofacial anatomy to secure the prosthetic device thereto.

9. The system of claim 1, wherein the controller device step of directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the surgical guide in the aligned relation with the maxillofacial anatomy, the surgical guide defining one or more guide holes or having one or more guide inserts engaged therewith, with the one or more guide inserts each defining a guide hole, so as to allow securement holes to be drilled in the maxillofacial anatomy through the guides holes or to allow fasteners to be received through the guide holes for engagement with the securement holes so as to secure the surgical guide to the maxillofacial anatomy.

10. The system of claim 1, wherein the prosthetic device defines one or more guide holes, or has one or more guide inserts engaged therewith, with the one or more guide inserts each defining a guide hole, such that the prosthetic device is the surgical guide.

11. The system of claim 1, wherein the orthognathic element is a portion of the maxillofacial anatomy separated from a remainder of the maxillofacial anatomy, and wherein the step of directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the portion of the maxillofacial anatomy in the aligned relation with the remainder of the maxillofacial anatomy, so as to allow securement holes to be drilled in the portion or the remainder of the maxillofacial anatomy or to allow fasteners to be engaged with the securement holes so as to secure a bracket between the portion and the remainder of the maxillofacial anatomy.

12. The system of claim 1, comprising a detector engaged with a distal end of a tracking arm, the tracking arm and the detector being in communication with the controller device, the detector being arranged in a spaced-apart relationship with the fiducial marker to detect the fiducial marker and cooperate with the controller device to determine a spatial relationship with the fiducial marker.

13. The system of claim 12, wherein the detector is an electrical detector, an electromechanical detector, an electromagnetic detector, an optical detector, an infrared detector, or combinations thereof.

14. The system of claim 1, comprising a tracking arm having a distal end physically engaged with the fiducial marker, the tracking arm being in communication with the controller device and arranged to cooperate with the controller device to determine a spatial relationship with the fiducial marker.

15. The system of claim 1, wherein the fiducial marker is arranged to be in communication with the controller device via an electrical communication system, a mechanical communication system, an electromechanical communication system, an electromagnetic communication system, an optical communication system, an infrared communication system, or combinations thereof.

16. The system of claim 1, wherein the fiducial marker is arranged to be in communication with the controller device via a wireless communication system or a wired communication system.

17. The system of claim 1, wherein the controller device is arranged to facilitate graphical manipulation of an image of the maxillofacial anatomy to form the virtual procedure plan for the maxillofacial anatomy receiving the prosthetic device, the surgical guide, or the orthognathic element in the aligned relation therewith.

18. A method of conducting a dental procedure implementing a dental robotic system, the method comprising:
engaging a fiducial marker with maxillofacial anatomy;
receiving a prosthetic device, a surgical guide, or an orthognathic element, in a predetermined spatial relationship with an end effector engaged with a distal end of an articulating arm member; and
executing a computer program product with a processor of a controller device to perform the steps of:
communicating with the fiducial marker, the articulating arm member, and the end effector, to determine a disposition of the end effector in relation to the fiducial marker during movement of the end effector in accordance with a virtual procedure plan for placing the prosthetic device, the surgical guide, or the orthognathic element in an aligned relation to the maxillofacial anatomy; and
directing the articulating arm member to physically control allowable movement of the end effector, directly relative to the disposition of the end effector with respect to the fiducial marker engaged with the maxillofacial anatomy, according to the virtual procedure plan, in order to place the prosthetic device, the surgical guide, or the orthognathic element in the aligned relation for securement to the maxillofacial anatomy.

19. The method of claim 18, wherein executing the computer program product with the processor of the controller device causes the controller device to perform the step of directing the end effector to provide haptic feedback, if movement of the end effector deviates from the virtual procedure plan, as the prosthetic device, the surgical guide, or the orthognathic element is being placed in the aligned relation to the maxillofacial anatomy.

20. The method of claim 18, wherein engaging a fiducial marker with the maxillofacial anatomy comprises physically and securely interacting a splint device with the maxillofacial anatomy, the splint device being operably engaged with the fiducial marker.

21. The method of claim 18, comprising arranging the controller device or the end effector to provide audio feedback or visual feedback, if movement of the end effector deviates from the virtual procedure plan.

22. The method of claim 18, comprising directly receiving the prosthetic device, the surgical guide, or the orthognathic element with the end effector, such that the prosthetic device, the surgical guide, or the orthognathic element is received in a predetermined spatial relationship therewith.

23. The method of claim 18, comprising receiving an interface with the end effector in a predetermined spatial relationship therewith, the interface being arranged to receive the prosthetic device, the surgical guide, or the orthognathic element in a predetermined spatial relationship therewith.

24. The method of claim 23, wherein the interface is a bite form appliance and wherein the method comprises receiving the prosthetic device with the bite form appliance in the predetermined spatial relationship therewith.

25. The method of claim 18, wherein directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the prosthetic device in the aligned relation with one or more implant anchors implanted in the maxillofacial anatomy to secure the prosthetic device thereto.

26. The method of claim 18, wherein directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the surgical guide in the aligned relation with the maxillofacial anatomy, the surgical guide defining one or more guide holes or having one or more guide inserts engaged therewith, with the one or more guide inserts each defining a guide hole, so as to allow securement holes to be drilled in the maxillofacial anatomy through the guides holes or to allow fasteners to be received through the guide holes for engagement with the securement holes so as to secure the surgical guide to the maxillofacial anatomy.

27. The method of claim 18, comprising forming one or more guide holes in the prosthetic device, or engaging one or more guide inserts with the prosthetic device, with the one or more guide inserts each defining a guide hole, such that the prosthetic device becomes the surgical guide.

28. The method of claim 18, wherein the orthognathic element is a portion of the maxillofacial anatomy separated from a remainder of the maxillofacial anatomy, and wherein directing the articulating arm member to physically control allowable movement of the end effector, comprises directing the articulating arm member to physically control allowable movement of the end effector, directly relative to the disposition of the end effector with respect to the fiducial marker engaged with the maxillofacial anatomy, according to the virtual procedure plan in order to place the portion of the maxillofacial anatomy in the aligned relation with the remainder of the maxillofacial anatomy, so as to allow securement holes to be drilled in the portion or the remainder of the maxillofacial anatomy or to allow fasteners to be engaged with the securement holes so as to secure a bracket between the portion and the remainder of the maxillofacial anatomy.

29. The method of claim 18, wherein communicating with the fiducial marker comprises communicating with the fiducial marker via a detector engaged with a distal end of a tracking arm, the tracking arm and the detector being in communication with the controller device, and the detector being arranged in a spaced-apart relationship with the fiducial marker to detect the fiducial marker and cooperate with the controller device to determine a spatial relationship with the fiducial marker.

30. The method of claim 18, wherein communicating with the fiducial marker comprises communicating with the fiducial marker via an electrical detector, an electromechanical detector, an electromagnetic detector, an optical detector, an infrared detector, or combinations thereof, engaged with the distal end of the tracking arm.

31. The method of claim 18, wherein communicating with the fiducial marker comprises communicating with the fiducial marker via a distal end of a tracking arm, the distal end being physically engaged with the fiducial marker, the tracking arm being in communication with the controller device and arranged to cooperate with the controller device to determine a spatial relationship with the fiducial marker.

32. The method of claim 18, comprising communicating between the fiducial marker and the controller device via an electrical communication system, a mechanical communication system, an electromechanical communication system, an electromagnetic communication system, an optical communication system, an infrared communication system, or combinations thereof.

33. The method of claim 18, comprising communicating between the fiducial marker and the controller device via a wireless communication system or a wired communication system.

34. The method of claim 18, comprising graphically manipulating an image of the maxillofacial anatomy with the controller device to form the virtual procedure plan for the maxillofacial anatomy receiving the prosthetic device, the surgical guide, or the orthognathic element in the aligned relation therewith.
